(19) United States
(12) Patent Application Publication
(10) Pub. No.: US 2024/0343712 A1
(43) Pub. Date: Oct. 17, 2024

(54) SOLID-STATE FORMS OF 2-(3-(4-(1H-INDAZOL-5-YLAMINO)QUINAZOLIN-2-YL)PHENOXY)-N-ISOPROPYLACETAMIDE METHANE SULFONIC ACID SALT

(71) Applicant: Sandoz AG, Basel (CH)

(72) Inventors: Hannes Lengauer, Kundl (AT); Renate Margreiter, Kundl (AT); Arthur Pichler, Kundl (AT)

(73) Assignee: Sandoz AG, Basel (CH)

(21) Appl. No.: 18/579,041

(22) PCT Filed: Jul. 18, 2022
(86) PCT No.: PCT/EP2022/070063

§ 371 Date: Jan. 12, 2024

(30) Foreign Application Priority Data
Jul. 16, 2021 (EP) 21186105.9
Sep. 29, 2021 (EP) 21199739.0

(51) Int. Cl. C07D 403/12; A61K 9/28; A61K 31/517

(52) U.S. Cl. CPC C07D 403/12; A61K 9/28; A61K 31/517

(57) ABSTRACT

The present invention relates to solid-state forms of 2-(3-(4-(1H-indazol-5-ylamino)quinazolin-2-yl)phenoxy)-N-isopropylacetamide methane sulfonic acid (INN: belumosudil mesylate) and processes for their preparation. Furthermore, the invention relates to a pharmaceutical composition comprising a solid-state form of the present invention and at least one pharmaceutically acceptable excipient. The pharmaceutical composition of the present invention can be used as a medicament, in particular for the treatment and/or prophylaxis of autoimmune diseases such as graft-versus-host disease (GvHD) and systemic sclerosis.

### FIELD OF THE INVENTION

The present invention relates to solid-state forms of 2-(3-(4-(1H-indazol-5-ylamino)quinazolin-2-yl)phenoxy)-N-isopropylacetamide methane sulfonic acid (INN: belumosudil mesylate) and processes for their preparation. Furthermore, the invention relates to a pharmaceutical composition comprising a solid-state form of the present invention and at least one pharmaceutically acceptable excipient. The pharmaceutical composition of the present invention can be used as a medicament, in particular for the treatment and/or prophylaxis of an autoimmune disease such as graft-versus-host disease (GvHD) and systemic sclerosis.

### BACKGROUND OF THE INVENTION

Graft-versus-host disease is a complication that can occur in patients who have had a transplant. In this disease, the transplanted cells recognise the patient's body as “foreign” and attack the patient's organs, such as the stomach, gut, skin and liver, leading to organ damage. The disease may occur shortly after transplantation or later on, in which case more organs can be affected. Graft-versus-host disease is a serious and life-threatening disease with a high mortality rate.

Belumosudil blocks an enzyme called Rho-associated protein kinase 2 (ROCK2) that plays a role in the inflammation that occurs in graft-versus-host disease and leads to organ damage. By blocking this enzyme, belumosudil is expected to help reduce inflammation and thereby relieve symptoms of the condition.

The chemical name of belumosudil is 2-(3-(4-(1H-indazol-5-ylamino)quinazolin-2-yl)phenoxy)-N-isopropylacetamide and it can be represented by the following chemical structure according to Formula (A)

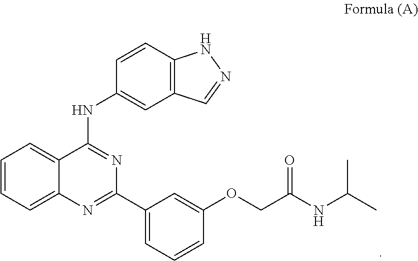 [SMILES: CC(C)NC(=O)COc1cccc(-c2nc(Nc3ccc4[nH]ncc4c3)c3ccccc3n2)c1]

Belumosudil and its preparation are disclosed in WO 2006/105081 A1. In the last step of example 82 crude belumosudil was purified using preparative HPLC. Example 92 of WO 2008/054599 A1 discloses the preparation of a belumosudil trifluoroacetate salt. The preparation of a belumosudil hydrochloride salt is disclosed in example 5 of WO 2014/055999 A1.

Different solid-state forms of an active pharmaceutical ingredient (API) often possess different properties. Differences in physicochemical properties of solid-state forms can play a crucial role for the improvement of pharmaceutical compositions, for example, pharmaceutical formulations with improved dissolution profile and bioavailability or with improved stability or shelf-life can become accessible due to an improved solid-state form of an active pharmaceutical ingredient. Also processing or handling of the active pharmaceutical ingredient during the formulation process may be improved. New solid-state forms of an active pharmaceutical ingredient can thus have desirable processing properties. They can be easier to handle, better suited for storage, and/or allow for better purification, compared to previously known solid-state forms.

The belumosudil trifluoroacetate salt of WO 2008/054599 A1 and the belumosudil hydrochloride salt of WO 2014/055999 A1 suffer from certain drawbacks which compromise their use in pharmaceutical compositions, particularly their use in solid dosage forms. For example, they show low crystallinity (see FIGS. 15 and 16 hereinafter) and significantly interact with water vapour upon moisture contact (see Comparative Example 1 herein). While the belumosudil hydrochloride salt takes up significantly high amounts of water with increasing relative humidity levels, the belumosudil trifluoroacetate salt contains unacceptable high levels of residual solvents which it partially releases and exchanges against water upon moisture contact.

There is thus a need for the provision of solid-state forms of belumosudil having improved physicochemical properties.

### SUMMARY OF THE INVENTION

The present invention provides crystalline forms of belumosudil mesylate, hereinafter also designated as “Form I”, “Form II”, “Form HyA” and “Form HyB” as well as an amorphous form of belumosudil mesylate.

The solid-state forms of the present invention possess one or more advantageous properties selected from the group consisting of chemical stability, physical stability, melting point, hygroscopicity, solubility, dissolution, morphology, crystallinity, flowability, bulk density, compactibility and wettability.

In particular, belumosudil mesylate Form I, Form II, Form HyA and Form HyB exhibit high crystallinity (see FIGS. 1 to 4 and 6). Moreover, anhydrous belumosudil mesylate Form I and Form II of the present invention take up less than 2 w-% of water when measured with gravimetric moisture sorption in the range of 0-80% relative humidity and a temperature of (25±0.1)° C. and can therefore be assigned as being only slightly hygroscopic. Moreover, both anhydrous belumosudil mesylate forms of the present invention are stable against temperature stress (see Examples 7 and 8 as well as FIGS. 7, 8, 11 and 12 hereinafter).

### Abbreviations

PXRD powder X-ray diffractogram
FTIR Fourier transform infrared
DSC differential scanning calorimetry
TGA thermogravimetric analysis
GMS gravimetric moisture sorption
w-% weight percent
RH relative humidity
TFA trifluoroacetic acid

### Definitions

In the context of the present invention the following definitions have the indicated meaning, unless explicitly stated otherwise:

As used herein, the term “measured at a temperature in the range of from 20 to 30° C.” refers to a measurement under standard conditions. Typically, standard conditions mean a temperature in the range of from 20 to 30° C., i.e. at room temperature. Standard conditions can mean a temperature of about 22° C. Typically, standard conditions can additionally mean a measurement at 20-60% RH, preferably at 30-50% RH, more preferably at 40% RH.

As used herein the term “room temperature” refers to a temperature in the range of from 20 to 30° C.

The term “reflection” with regard to powder X-ray diffraction as used herein, means peaks in an X-ray diffractogram, which are caused at certain diffraction angles (Bragg angles) by constructive interference from X-rays scattered by parallel planes of atoms in solid material, which are distributed in an ordered and repetitive pattern in a long-range positional order. Such a solid material is classified as crystalline material, whereas amorphous material is defined as solid material, which lacks long-range order and only displays short-range order, thus resulting in broad scattering. According to literature, long-range order e.g. extends over approximately 100 to 1000 atoms, whereas short-range order is over a few atoms only (see “Fundamentals of Powder Diffraction and Structural Characterization of Materials” by Vitalij K. Pecharsky and Peter Y. Zavalij, Kluwer Academic Publishers, 2003, page 3).

The term “essentially the same” with reference to powder X-ray diffraction means that variabilities in reflection positions and relative intensities of the reflections are to be taken into account. For example, a typical precision of the 2-Theta values is in the range of ±0.2° 2-Theta, preferably of ±0.1° 2-Theta. Thus, a reflection that usually appears at 7.1° 2-Theta for example can appear between 6.9° and 7.3° 2-Theta, preferably between 7.0° and 7.2° 2-Theta on most X-ray diffractometers under standard conditions. Furthermore, one skilled in the art will appreciate that relative reflection intensities will show inter-apparatus variability as well as variability due to degree of crystallinity, preferred orientation, particle size, sample preparation and other factors known to those skilled in the art and should be taken as qualitative measure only.

A solid-state form of belumosudil mesylate of the present invention may be referred to herein as being characterized by graphical data “as shown in” a figure. Such data include, for example, powder X-ray diffraction. The person skilled in the art understands that factors such as variations in instrument type, response and variations in sample directionality, sample concentration and sample purity may lead to small variations for such data when presented in graphical form, for example variations relating to the exact reflection positions and intensities. However, a comparison of the graphical data in the figures herein with the graphical data generated for another or an unknown solid form and the confirmation that two sets of graphical data relate to the same crystal form is well within the knowledge of a person skilled in the art.

The term “solid-state form” as used herein refers to any crystalline and/or amorphous phase of a compound.

As used herein, the term “amorphous” refers to a solid-state form of a compound that is not crystalline. An amorphous compound possesses no long-range order and does not display a definitive X-ray diffraction pattern with reflections.

The term “anhydrous” as used herein refers to a crystalline solid where no water is cooperated in or accommodated by the crystal structure.

A “predetermined amount” as used herein with regard to a crystalline or amorphous form of belumosudil mesylate refers to the initial amount of the form calculated as belumosudil used for the preparation of a pharmaceutical composition having a desired dosage strength of belumosudil.

The term “effective amount” as used herein with regard to a crystalline or amorphous form of belumosudil mesylate encompasses an amount of the form calculated as belumosudil which causes the desired therapeutic and/or prophylactic effect.

As used herein, the term “about” means within a statistically meaningful range of a value. Such a range can be within an order of magnitude, typically within 10%, more typically within 5%, even more typically within 1% and most typically within 0.1% of the indicated value or range. Sometimes, such a range can lie within the experimental error, typical of standard methods used for the measurement and/or determination of a given value or range.

The term “pharmaceutically acceptable excipient” as used herein refers to substances, which do not show a significant pharmacological activity at the given dose and that are added to a pharmaceutical composition in addition to the active pharmaceutical ingredient. Excipients may take the function of vehicle, diluent, release agent, disintegrating agent, dissolution modifying agent, absorption enhancer, stabilizer or a manufacturing aid among others.

The term “filler” as used herein refers to substances that are used to dilute the active pharmaceutical ingredient prior to delivery. Fillers can also serve as stabilizer or disintegrant.

The term “disintegrant” as used herein refers to substances which, upon addition to a solid pharmaceutical composition, facilitate its break-up or disintegration after administration and permits the release of the active pharmaceutical ingredient as efficiently as possible to allow for its rapid dissolution.

The term “lubricant” as used herein refers to substances which are added to a powder blend to prevent the compacted powder mass from sticking to the equipment during tableting or encapsulation process.

As used herein the term “binder” refers to substances which bind the active pharmaceutical ingredient and pharmaceutically acceptable excipient together to maintain cohesive and discrete portions.

The term “glidant” as used herein refers to substances which are used for tablet and capsule formulations in order to improve flow properties during tablet compression and to produce an anti-caking effect.

### BRIEF DESCRIPTION OF THE FIGURES

FIG. 1: illustrates a representative PXRD of belumosudil mesylate Form I according to the present invention. The x-axis shows the scattering angle in °2-Theta, the y-axis shows the intensity of the scattered X-ray beam in counts of detected photons.

FIG. 2: illustrates a representative PXRD of belumosudil mesylate Form II according to the present invention. The x-axis shows the scattering angle in °2-Theta, the y-axis shows the intensity of the scattered X-ray beam in counts of detected photons.

FIG. 3: illustrates a representative PXRD of belumosudil mesylate Form HyA according to the present invention. The x-axis shows the scattering angle in °2-Theta, the y-axis shows the intensity of the scattered X-ray beam in counts of detected photons.

FIG. 4: illustrates a representative PXRD of belumosudil mesylate Form HyB according to the present invention. The x-axis shows the scattering angle in °2-Theta, the y-axis shows the intensity of the scattered X-ray beam in counts of detected photons.

FIG. 5: illustrates a representative PXRD of amorphous belumosudil mesylate. The x-axis shows the scattering angle in °2-Theta, the y-axis shows the intensity of the scattered X-ray beam in counts of detected photons.

FIG. 6: illustrates a comparison of PXRDs of belumosudil mesylate Form I, Form II, Form HyA and Form HyB (from top to bottom). The x-axis shows the scattering angle in °2-Theta. The PXRDs were shifted along the y-axis to separate the diffractograms for clarity.

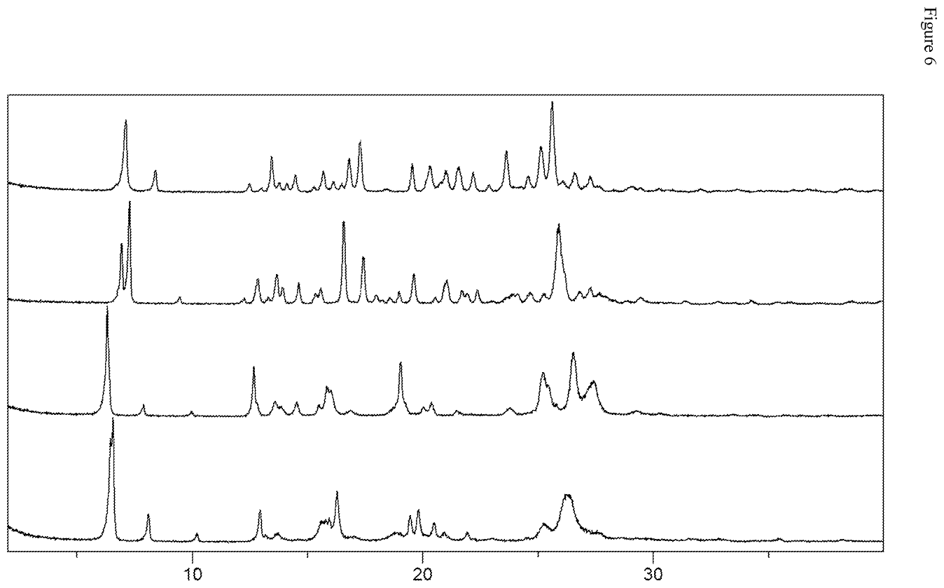

FIG. 7: illustrates a representative DSC curve of belumosudil mesylate Form I according to the present invention. The x-axis shows the temperature in degree Celsius (° C.), the y-axis shows the heat flow rate in Watt per gram (W/g) with endothermic peaks going up.

FIG. 8: illustrates a representative DSC curve of belumosudil mesylate Form II according to the present invention. The x-axis shows the temperature in degree Celsius (° C.), the y-axis shows the heat flow rate in Watt per gram (W/g) with endothermic peaks going up.

FIG. 9: illustrates a representative DSC curve of belumosudil mesylate Form HyA according to the present invention. The x-axis shows the temperature in degree Celsius (° C.), the y-axis shows the heat flow rate in Watt per gram (W/g) with endothermic peaks going up.

FIG. 10: illustrates a representative DSC curve of belumosudil mesylate Form HyB according to the present invention. The x-axis shows the temperature in degree Celsius (° C.), the y-axis shows the heat flow rate in Watt per gram (W/g) with endothermic peaks going up.

FIG. 11: illustrates a representative TGA curve of belumosudil mesylate Form I of the present invention. The x-axis shows the temperature in degree Celsius (° C.), the y-axis shows the mass (loss) of the sample in weight percent (w-%).

FIG. 12: illustrates a representative TGA curve of belumosudil mesylate Form II of the present invention. The x-axis shows the temperature in degree Celsius (° C.), the y-axis shows the mass (loss) of the sample in weight percent (w-%).

FIG. 13: illustrates a representative TGA curve of belumosudil mesylate Form HyA of the present invention. The x-axis shows the temperature in degree Celsius (° C.), the y-axis shows the mass (loss) of the sample in weight percent (w-%).

FIG. 14: illustrates a representative TGA curve of belumosudil mesylate Form HyB of the present invention. The x-axis shows the temperature in degree Celsius (° C.), the y-axis shows the mass (loss) of the sample in weight percent (w-%).

FIG. 15: illustrates a representative PXRD of the belumosudil HCl salt prepared according to the procedure disclosed in Example 5 of WO 2014/055999 A1. The x-axis shows the scattering angle in °2-Theta, the y-axis shows the intensity of the scattered X-ray beam in counts of detected photons.

FIG. 16: illustrates a representative PXRD of the belumosudil TFA salt prepared according to the procedure disclosed in Example 92 of WO 2008/054599 A1. The x-axis shows the scattering angle in °2-Theta, the y-axis shows the intensity of the scattered X-ray beam in counts of detected photons.

### DETAILED DESCRIPTION OF THE INVENTION

The present invention relates to solid-state forms of belumosudil mesylate including crystalline belumosudil mesylate and forms as well as amorphous belumosudil mesylate.

Hence, in a first aspect the present invention relates to crystalline belumosudil mesylate according to the chemical structure as depicted in Formula (B)

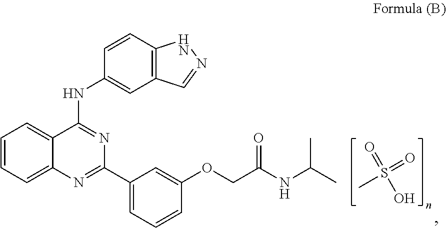

wherein n is in the range of from 0.8 to 1.2, preferably of from 0.9 to 1.1, even more preferably of from 0.95 to 1.05 and most preferably n is 1.0.

In particular the present invention relates to crystalline forms of belumosudil mesylate herein also designated as “Form I”, “Form II”, Form HyA” and “Form HyB”. The crystalline forms of belumosudil mesylate of the present invention may be characterized by analytical methods well known in the field of the pharmaceutical industry for characterizing solids. Such methods comprise but are not limited to powder X-ray diffraction, single X-ray diffraction, FTIR spectroscopy, DSC, TGA and GMS. The crystalline forms of the present invention may be characterized by one of the aforementioned analytical methods or by combining two or more of them. In particular, the crystalline forms of belumosudil mesylate of the present invention may be characterized by any one of the following embodiments or by combining two or more of the following embodiments.

### Crystalline Form I of Belumosudil Mesylate

In one embodiment the invention relates to a crystalline form (Form I) of belumosudil mesylate characterized by having a PXRD comprising reflections at 2-Theta angles of:
(7.1±0.2)°, (8.4±0.2)° and (25.6±0.2)°; or
(7.1±0.2)°, (8.4±0.2)°, (20.3±0.2)° and (25.6±0.2)°; or
(7.1±0.2)°, (8.4±0.2)°, (17.3±0.2)°, (20.3±0.2)° and (25.6±0.2)°; or
(7.1±0.2)°, (8.4±0.2)°, (13.4±0.2)°, (17.3±0.2)°, (20.3±0.2)° and (25.6±0.2)°; or
(7.1±0.2)°, (8.4±0.2)°, (13.4±0.2)°, (17.3±0.2)°, (20.3±0.2)°, (23.6±0.2)° and (25.6±0.2)°; or
(7.1±0.2)°, (8.4±0.2)°, (13.4±0.2)°, (17.3±0.2)°, (19.5±0.2)°, (20.3±0.2)°, (23.6±0.2)° and (25.6±0.2)°; or
(7.1±0.2)°, (8.4±0.2)°, (13.4±0.2)°, (16.8±0.2)°, (17.3±0.2)°, (19.5±0.2)°, (20.3±0.2)°, (23.6±0.2)° and (25.6±0.2)°; or
(7.1±0.2)°, (8.4±0.2)°, (13.4±0.2)°, (16.8±0.2)°, (17.3±0.2)°, (19.5±0.2)°, (20.3±0.2)°, (21.6±0.2)°, (23.6±0.2)° and (25.6±0.2)°; or
(7.1±0.2)°, (8.4±0.2)°, (13.4±0.2)°, (15.7±0.2)°, (16.8±0.2)°, (17.3±0.2)°, (19.5±0.2)°, (20.3±0.2)°, (21.6±0.2)°, (23.6±0.2)° and (25.6±0.2)°; or
(7.1±0.2)°, (8.4±0.2)°, (13.4±0.2)°, (14.5±0.2)°, (15.7±0.2)°, (16.8±0.2)°, (17.3±0.2)°, (19.5±0.2)°, (20.3±0.2)°, (21.6±0.2)°, (23.6±0.2)° and (25.6±0.2)°; or
(7.1±0.2)°, (8.4±0.2)°, (12.5±0.2)°, (13.4±0.2)°, (14.5±0.2)°, (15.7±0.2)°, (16.8±0.2)°, (17.3±0.2)°, (19.5±0.2)°, (20.3±0.2)°, (21.6±0.2)°, (23.6±0.2)° and (25.6±0.2)°; or
(7.1±0.2)°, (8.4±0.2)°, (12.5±0.2)°, (13.4±0.2)°, (14.5±0.2)°, (15.7±0.2)°, (16.8±0.2)°, (17.3±0.2)°, (19.5±0.2)°, (20.3±0.2)°, (21.6±0.2)°, (23.6±0.2)°, (25.1±0.2)° and (25.6±0.2)°; or
(7.1±0.2)°, (8.4±0.2)°, (12.5±0.2)°, (13.4±0.2)°, (14.5±0.2)°, (15.7±0.2)°, (16.8±0.2)°, (17.3±0.2)°, (19.5±0.2)°, (20.3±0.2)°, (21.6±0.2)°, (22.2±0.2)°, (23.6±0.2)°, (25.1±0.2)° and (25.6±0.2)°; or
(7.1±0.2)°, (8.4±0.2)°, (12.5±0.2)°, (13.4±0.2)°, (14.5±0.2)°, (15.7±0.2)°, (16.8±0.2)°, (17.3±0.2)°, (19.5±0.2)°, (20.3±0.2)°, (21.0±0.2)°, (21.6±0.2)°, (22.2±0.2)°, (23.6±0.2)°, (25.1±0.2)° and (25.6±0.2)°;
when measured at a temperature in the range of from 20 to 30° C. with Cu-Kalpha_{1,2} radiation having a wavelength of 0.15419 nm.

In another embodiment, the invention relates to a crystalline form (Form I) of belumosudil mesylate characterized by having a PXRD comprising reflections at 2-Theta angles of:
(7.1±0.1)°, (8.4±0.1)° and (25.6±0.1)°; or
(7.1±0.1)°, (8.4±0.1)°, (20.3±0.1)° and (25.6±0.1)°; or
(7.1±0.1)°, (8.4±0.1)°, (17.3±0.1)°, (20.3±0.1)° and (25.6±0.1)°; or
(7.1±0.1)°, (8.4±0.1)°, (13.4±0.1)°, (17.3±0.1)°, (20.3±0.1)° and (25.6±0.1)°; or
(7.1±0.1)°, (8.4±0.1)°, (13.4±0.1)°, (17.3±0.1)°, (20.3±0.1)°, (23.6±0.1)° and (25.6±0.1)°; or
(7.1±0.1)°, (8.4±0.1)°, (13.4±0.1)°, (17.3±0.1)°, (19.5±0.1)°, (20.3±0.1)°, (23.6±0.1)° and (25.6±0.1)°; or
(7.1±0.1)°, (8.4±0.1)°, (13.4±0.1)°, (16.8±0.1)°, (17.3±0.1)°, (19.5±0.1)°, (20.3±0.1)°, (23.6±0.1)° and (25.6±0.1)°; or
(7.1±0.1)°, (8.4±0.1)°, (13.4±0.1)°, (16.8±0.1)°, (17.3±0.1)°, (19.5±0.1)°, (20.3±0.1)°, (21.6±0.1)°, (23.6±0.1)° and (25.6±0.1)°; or
(7.1±0.1)°, (8.4±0.1)°, (13.4±0.1)°, (15.7±0.1)°, (16.8±0.1)°, (17.3±0.1)°, (19.5±0.1)°, (20.3±0.1)°, (21.6±0.1)°, (23.6±0.1)° and (25.6±0.1)°; or
(7.1±0.1)°, (8.4±0.1)°, (13.4±0.1)°, (14.5±0.1)°, (15.7±0.1)°, (16.8±0.1)°, (17.3±0.1)°, (19.5±0.1)°, (20.3±0.1)°, (21.6±0.1)°, (23.6±0.1)° and (25.6±0.1)°; or
(7.1±0.1)°, (8.4±0.1)°, (12.5±0.1)°, (13.4±0.1)°, (14.5±0.1)°, (15.7±0.1)°, (16.8±0.1)°, (17.3±0.1)°, (19.5±0.1)°, (20.3±0.1)°, (21.6±0.1)°, (23.6±0.1)° and (25.6±0.1)°; or
(7.1±0.1)°, (8.4±0.1)°, (12.5±0.1)°, (13.4±0.1)°, (14.5±0.1)°, (15.7±0.1)°, (16.8±0.1)°, (17.3±0.1)°, (19.5±0.1)°, (20.3±0.1)°, (21.6±0.1)°, (23.6±0.1)°, (25.1±0.1)° and (25.6±0.1)°; or
(7.1±0.1)°, (8.4±0.1)°, (12.5±0.1)°, (13.4±0.1)°, (14.5±0.1)°, (15.7±0.1)°, (16.8±0.1)°, (17.3±0.1)°, (19.5±0.1)°, (20.3±0.1)°, (21.6±0.1)°, (22.2±0.1)°, (23.6±0.1)°, (25.1±0.1)° and (25.6±0.1)°; or
(7.1±0.1)°, (8.4±0.1)°, (12.5±0.1)°, (13.4±0.1)°, (14.5±0.1)°, (15.7±0.1)°, (16.8±0.1)°, (17.3±0.2)°, (19.5±0.1)°, (20.3±0.1)°, (21.0±0.1)°, (21.6±0.1)°, (22.2±0.1)°, (23.6±0.1)°, (25.1±0.1)° and (25.6±0.1)°;
when measured at a temperature in the range of from 20 to 30° C. with Cu-Kalpha_{1,2} radiation having a wavelength of 0.15419 nm.

In a further embodiment, the present invention relates to a crystalline form (Form I) of belumosudil mesylate characterized by having a PXRD comprising reflections at 2-Theta angles of (7.1±0.2)°, (13.4±0.2)°, (16.8±0.2)°, (17.3±0.2)°, (19.5±0.2)°, (20.3±0.2)°, (21.6±0.2)°, (23.6±0.2)°, (25.1±0.2)° and (25.6±0.2)°, when measured at a temperature in the range of from 20 to 30° C. with Cu-Kalpha_{1,2} radiation having a wavelength of 0.15419 nm.

In still another embodiment, the present invention relates to a crystalline form (Form I) of belumosudil mesylate characterized by having a PXRD comprising reflections at 2-Theta angles of (7.1±0.1)°, (13.4±0.1)°, (16.8±0.1)°, (17.3±0.1)°, (19.5±0.1)°, (20.3±0.1)°, (21.6±0.1)°, (23.6±0.1)°, (25.1±0.1)° and (25.6±0.1)°, when measured at a temperature in the range of from 20 to 30° C. with Cu-Kalpha_{1,2} radiation having a wavelength of 0.15419 nm.

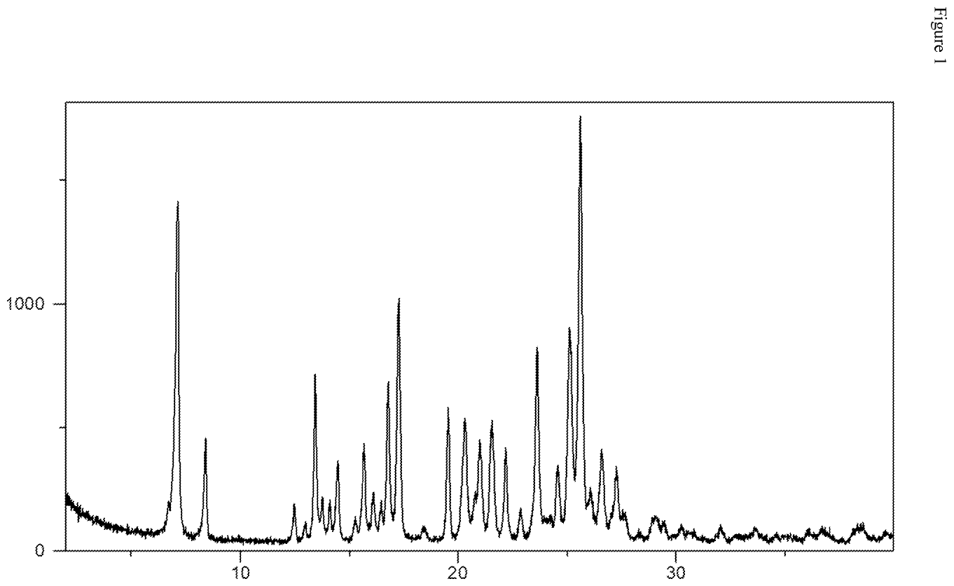

In yet another embodiment, the present invention relates to a crystalline form (Form I) of belumosudil mesylate characterized by having a PXRD essentially the same as shown in FIG. 1 of the present invention, when measured at a temperature in the range of from 20 to 30° C. with Cu-Kalpha_{1,2} radiation having a wavelength of 0.15419 nm.

In another embodiment, the present invention relates to a crystalline form (Form I) of belumosudil mesylate, characterized by having a DSC curve comprising an endothermic peak, preferably a single endothermic peak, having an onset at a temperature of about (264±5)° C., preferably of about (264±3)° C., more preferably of about (264±1)° C., such as at 263.9° C., when measured at a heating rate of 10 K/min.

In a further embodiment, the present invention relates to a crystalline form (Form I) of belumosudil mesylate as defined in any one of the above described embodiments characterized in being anhydrous.

### Crystalline Form II of Belumosudil Mesylate

In one embodiment the invention relates to a crystalline form (Form II) of belumosudil mesylate characterized by having a PXRD comprising reflections at 2-Theta angles of:
(7.3±0.2)°, (9.4±0.2)° and (16.6±0.2)°; or
(6.9±0.2)°, (7.3±0.2)°, (9.4±0.2)° and (16.6±0.2)°; or
(6.9±0.2)°, (7.3±0.2)°, (9.4±0.2)°, (16.6±0.2)° and (25.9±0.2)°; or
(6.9±0.2)°, (7.3±0.2)°, (9.4±0.2)°, (16.6±0.2)°, (17.4±0.2)° and (25.9±0.2)°; or
(6.9±0.2)°, (7.3±0.2)°, (9.4±0.2)°, (14.6±0.2)°, (16.6±0.2)°, (17.4±0.2)° and (25.9±0.2)°; or
(6.9±0.2)°, (7.3±0.2)°, (9.4±0.2)°, (14.6±0.2)°, (16.6±0.2)°, (17.4±0.2)°, (19.6±0.2)° and (25.9±0.2)°; or
(6.9±0.2)°, (7.3±0.2)°, (9.4±0.2), (13.7±0.2)°, (14.6±0.2)°, (16.6±0.2)°, (17.4±0.2)°, (19.6±0.2)° and (25.9±0.2)°; or
(6.9±0.2)°, (7.3±0.2)°, (9.4±0.2)°, (12.8±0.2)°, (13.7±0.2)°, (14.6±0.2)°, (16.6±0.2)°, (17.4±0.2)°, (19.6±0.2)° and (25.9±0.2)°;
when measured at a temperature in the range of from 20 to 30° C. with Cu-Kalpha_{1,2} radiation having a wavelength of 0.15419 nm.

In another embodiment the invention relates to a crystalline form (Form II) of belumosudil mesylate characterized by having a PXRD comprising reflections at 2-Theta angles of:
(7.3±0.1)°, (9.4±0.1)° and (16.6±0.1)°; or
(6.9±0.1)°, (7.3±0.1)°, (9.4±0.1)° and (16.6±0.1)°; or
(6.9±0.1)°, (7.3±0.1)°, (9.4±0.1)°, (16.6±0.1)° and (25.9±0.1)°; or
(6.9±0.1)°, (7.3±0.1)°, (9.4±0.1)°, (16.6±0.1)°, (17.4±0.1)° and (25.9±0.1)°; or
(6.9±0.1)°, (7.3±0.1)°, (9.4±0.1)°, (14.6±0.1)°, (16.6±0.1)°, (17.4±0.1)° and (25.9±0.1)°; or
(6.9±0.1)°, (7.3±0.1)°, (9.4±0.1)°, (14.6±0.1)°, (16.6±0.1)°, (17.4±0.1)°, (19.6±0.1)° and (25.9±0.1)°; or
(6.9±0.1)°, (7.3±0.1)°, (9.4±0.1)°, (13.7±0.1)°, (14.6±0.1)°, (16.6±0.1)°, (17.4±0.1)°, (19.6±0.1)° and (25.9±0.1)°; or
(6.9±0.1)°, (7.3±0.1)°, (9.4±0.1)°, (12.8±0.1)°, (13.7±0.1)°, (14.6±0.1)°, (16.6±0.1)°, (17.4±0.1)°, (19.6±0.1)° and (25.9±0.1)°;
when measured at a temperature in the range of from 20 to 30° C. with Cu-Kalpha_{1,2} radiation having a wavelength of 0.15419 nm.

In a further embodiment, the present invention relates to a crystalline form (Form II) of belumosudil mesylate characterized by having a PXRD comprising reflections at 2-Theta angles of (6.9±0.2)°, (7.3±0.2)°, (12.8±0.2)°, (13.7±0.2)°, (14.6±0.2)°, (16.6±0.2)°, (17.4±0.2)°, (19.6±0.2)°, (21.0±0.2)° and (25.9±0.2)°, when measured at a temperature in the range of from 20 to 30° C. with Cu-Kalpha_{1,2} radiation having a wavelength of 0.15419 nm.

In still another embodiment, the present invention relates to a crystalline form (Form II) of belumosudil mesylate characterized by having a PXRD comprising reflections at 2-Theta angles of (6.9±0.1)°, (7.3±0.1)°, (12.8±0.1)°, (13.7±0.1)°, (14.6±0.1)°, (16.6±0.1)°, (17.4±0.1)°, (19.6±0.1)°, (21.0±0.1)° and (25.9±0.1)°, when measured at a temperature in the range of from 20 to 30° C. with Cu-Kalpha_{1,2} radiation having a wavelength of 0.15419 nm.

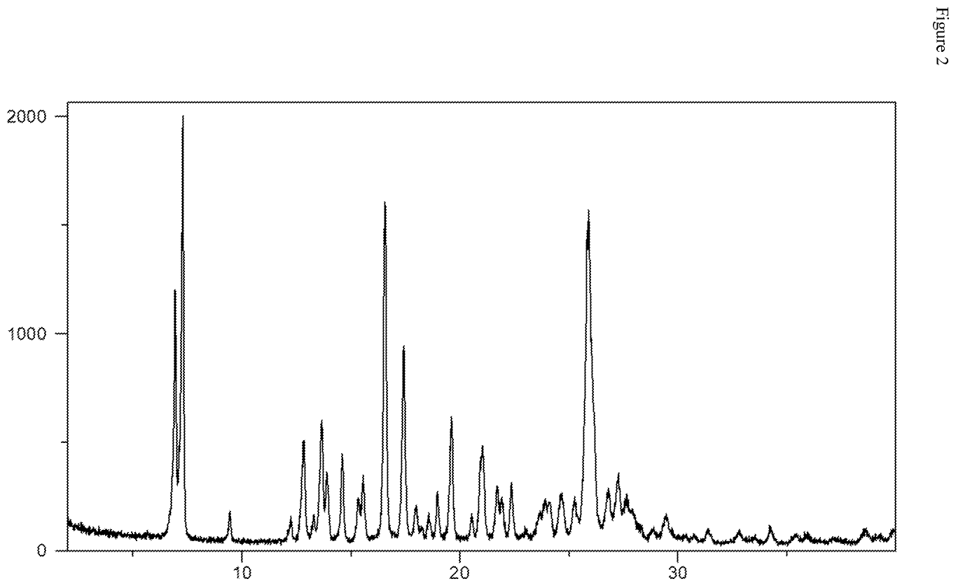

In yet another embodiment, the present invention relates to a crystalline form (Form II) of belumosudil mesylate characterized by having a PXRD essentially the same as shown in FIG. 2 of the present invention, when measured at a temperature in the range of from 20 to 30° C. with Cu-Kalpha_{1,2} radiation having a wavelength of 0.15419 nm.

In another embodiment, the present invention relates to a crystalline form (Form II) of belumosudil mesylate, characterized by having a DSC curve comprising an endothermic peak, preferably a single endothermic peak, having an onset at a temperature of about (248±5)° C., preferably of about (248±3)° C., more preferably of about (248±1)° C., such as at 247.5° C., when measured at a heating rate of 10 K/min.

In a further embodiment, the present invention relates to a crystalline form (Form II) of belumosudil mesylate as defined in any one of the above described embodiments characterized in being anhydrous.

### Crystalline Form HyA of Belumosudil Mesylate

In one embodiment the invention relates to a crystalline form (Form HyA) of belumosudil mesylate characterized by having a PXRD comprising reflections at 2-Theta angles of:
(6.3±0.2)°, (12.7±0.2)° and (14.5±0.2)°; or
(6.3±0.2)°, (12.7±0.2)°, (14.5±0.2)° and (19.0±0.2)°; or
(6.3±0.2)°, (7.9±0.2)°, (12.7±0.2)°, (14.5±0.2)° and (19.0±0.2)°; or
(6.3±0.2)°, (7.9±0.2)°, (10.0±0.2)°, (12.7±0.2)°, (14.5±0.2)° and (19.0±0.2)°; or
(6.3±0.2)°, (7.9±0.2)°, (10.0±0.2)°, (12.7±0.2)°, (14.5±0.2)°, (19.0±0.2)° and (26.5±0.2)°; or
(6.3±0.2)°, (7.9±0.2)°, (10.0±0.2)°, (12.7±0.2)°, (14.5±0.2)°, (19.0±0.2)°, (26.5±0.2)° and (27.4±0.2)°; or
(6.3±0.2)°, (7.9±0.2)°, (10.0±0.2)°, (12.7±0.2)°, (14.5±0.2)°, (15.8±0.2)°, (19.0±0.2)°, (26.5±0.2)° and (27.4±0.2)°; or
(6.3±0.2)°, (7.9±0.2)°, (10.0±0.2)°, (12.7±0.2)°, (13.6±0.2)°, (14.5±0.2)°, (15.8±0.2)°, (19.0±0.2)°, (26.5±0.2)° and (27.4±0.2)°;
when measured at a temperature in the range of from 20 to 30° C. with Cu-Kalpha_{1,2} radiation having a wavelength of 0.15419 nm.

In another embodiment the invention relates to a crystalline form (Form HyA) of belumosudil mesylate characterized by having a PXRD comprising reflections at 2-Theta angles of:
(6.3±0.1)°, (12.7±0.1)° and (14.5±0.1)°; or
(6.3±0.1)°, (12.7±0.1)°, (14.5±0.1)° and (19.0±0.1)°; or
(6.3±0.1)°, (7.9±0.1)°, (12.7±0.1)°, (14.5±0.1)° and (19.0±0.1)°; or
(6.3±0.1)°, (7.9±0.1)°, (10.0±0.1)°, (12.7±0.1)°, (14.5±0.1)° and (19.0±0.1)°; or
(6.3±0.1)°, (7.9±0.1)°, (10.0±0.1)°, (12.7±0.1)°, (14.5±0.1)°, (19.0±0.1)° and (26.5±0.1)°; or
(6.3±0.1)°, (7.9±0.1)°, (10.0±0.1)°, (12.7±0.1)°, (14.5±0.1)°, (19.0±0.1)°, (26.5±0.1)° and (27.4±0.1)°; or
(6.3±0.1)°, (7.9±0.1)°, (10.0±0.1)°, (12.7±0.1)°, (14.5±0.1)°, (15.8±0.1)°, (19.0±0.1)°, (26.5±0.1)° and (27.4±0.1)°; or
(6.3±0.1)°, (7.9±0.1)°, (10.0±0.1)°, (12.7±0.1)°, (13.6±0.1)°, (14.5±0.1)°, (15.8±0.1)°, (19.0±0.1)°, (26.5±0.1)° and (27.4±0.1)°;
when measured at a temperature in the range of from 20 to 30° C. with Cu-Kalpha_{1,2} radiation having a wavelength of 0.15419 nm.

In a further embodiment, the present invention relates to a crystalline form (Form HyA) of belumosudil mesylate characterized by having a PXRD comprising reflections at 2-Theta angles of (6.3±0.2)°, (12.7±0.2)°, (13.6±0.2)°, (15.8±0.2)°, (16.0±0.2)°, (19.0±0.2)°, (25.2±0.2)°, (25.4±0.2)°, (26.5±0.2)° and (27.4±0.2)°, when measured at a temperature in the range of from 20 to 30° C. with Cu-Kalpha_{1,2} radiation having a wavelength of 0.15419 nm.

In still another embodiment, the present invention relates to a crystalline form (Form HyA) of belumosudil mesylate characterized by having a PXRD comprising reflections at 2-Theta angles of (6.3±0.1)°, (12.7±0.1)°, (13.6±0.1)°, (15.8±0.1)°, (16.0±0.1)°, (19.0±0.1)°, (25.2±0.1)°, (25.4±0.1)°, (26.5±0.1)° and (27.4±0.1)°, when measured at a temperature in the range of from 20 to 30° C. with Cu-Kalpha_{1,2} radiation having a wavelength of 0.15419 nm.

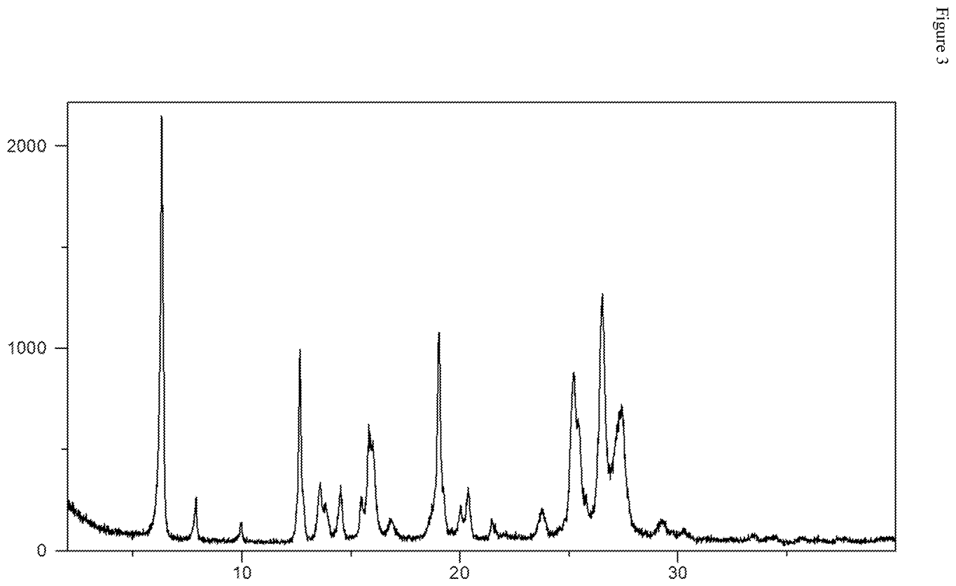

In yet another embodiment, the present invention relates to a crystalline form (Form HyA) of belumosudil mesylate characterized by having a PXRD essentially the same as shown in FIG. 3 of the present invention, when measured at a temperature in the range of from 20 to 30° C. with Cu-Kalpha_{1,2} radiation having a wavelength of 0.15419 nm.

In another embodiment, the present invention relates to a crystalline form (Form HyA) of belumosudil mesylate, characterized by having a DSC curve comprising an endothermic peak, having an onset at a temperature of about (76±5)° C., preferably of about (76±3)° C., more preferably of about (76±1)° C., such as 76.3° C., when measured at a heating rate of 10 K/min.

In a further embodiment, the present invention relates to a crystalline form (Form HyA) of belumosudil mesylate as defined in any one of the above described embodiments characterized in being a dihydrate.

### Crystalline Form HyB of Belumosudil Mesylate

In one embodiment the invention relates to a crystalline form (Form HyB) of belumosudil mesylate characterized by having a PXRD comprising reflections at 2-Theta angles of:
(6.6±0.2)°, (16.3±0.2)° and (19.5±0.2)°; or
(6.4±0.2)°, (6.6±0.2)°, (16.3±0.2)° and (19.5±0.2)°; or
(6.4±0.2)°, (6.6±0.2)°, (8.1±0.2)°, (16.3±0.2)° and (19.5±0.2)°; or
(6.4±0.2)°, (6.6±0.2)°, (8.1±0.2)°, (12.9±0.2)°, (16.3±0.2)° and (19.5±0.2)°; or
(6.4±0.2)°, (6.6±0.2)°, (8.1±0.2)°, (10.2±0.2)°, (12.9±0.2)°, (16.3±0.2)° and (19.5±0.2)°; or
(6.4±0.2)°, (6.6±0.2)°, (8.1±0.2)°, (10.2±0.2)°, (12.9±0.2)°, (16.3±0.2)°, (19.5±0.2)° and (19.8±0.2)°; or
(6.4±0.2)°, (6.6±0.2)°, (8.1±0.2)°, (10.2±0.2)°, (12.9±0.2)°, (16.3±0.2)°, (19.5±0.2)°, (19.8±0.2)° and (20.5±0.2)°; or
(6.4±0.2)°, (6.6±0.2)°, (8.1±0.2)°, (10.2±0.2)°, (12.9±0.2)°, (16.3±0.2)°, (19.5±0.2)°, (19.8±0.2)°, (20.5±0.2)° and (20.9±0.2)°;
when measured at a temperature in the range of from 20 to 30° C. with Cu-Kalpha_{1,2} radiation having a wavelength of 0.15419 nm.

In another embodiment the invention relates to a crystalline form (Form HyB) of belumosudil mesylate characterized by having a PXRD comprising reflections at 2-Theta angles of:
(6.6±0.1)°, (16.3±0.1)° and (19.5±0.1)°; or
(6.4±0.1)°, (6.6±0.1)°, (16.3±0.1)° and (19.5±0.1)°; or
(6.4±0.1)°, (6.6±0.1)°, (8.1±0.1)°, (16.3±0.1)° and (19.5±0.1)°; or
(6.4±0.1)°, (6.6±0.1)°, (8.1±0.1)°, (12.9±0.1)°, (16.3±0.1)° and (19.5±0.1)°; or
(6.4±0.1)°, (6.6±0.1)°, (8.1±0.1)°, (10.2±0.1)°, (12.9±0.1)°, (16.3±0.1)° and (19.5±0.1)°; or
(6.4±0.1)°, (6.6±0.1)°, (8.1±0.1)°, (10.2±0.1)°, (12.9±0.1)°, (16.3±0.1)°, (19.5±0.1)° and (19.8±0.1)°; or
(6.4±0.1)°, (6.6±0.1)°, (8.1±0.1)°, (10.2±0.1)°, (12.9±0.1)°, (16.3±0.1)°, (19.5±0.1)°, (19.8±0.1)° and (20.5±0.1)°; or
(6.4±0.1)°, (6.6±0.1)°, (8.1±0.1)°, (10.2±0.1)°, (12.9±0.1)°, (16.3±0.1)°, (19.5±0.1)°, (19.8±0.1)°, (20.5±0.1)° and (20.9±0.1)°;
when measured at a temperature in the range of from 20 to 30° C. with Cu-Kalpha_{1,2} radiation having a wavelength of 0.15419 nm.

In a further embodiment, the present invention relates to a crystalline form (Form HyB) of belumosudil mesylate characterized by having a PXRD comprising reflections at 2-Theta angles of (6.4±0.2)°, (6.6±0.2)°, (8.1±0.2)°, (12.9±0.2)°, (15.9±0.2)°, (16.3±0.2)°, (19.5±0.2)°, (19.8±0.2)°, (26.3±0.2)° and (26.4±0.2)°, when measured at a temperature in the range of from 20 to 30° C. with Cu-Kalpha_{1,2} radiation having a wavelength of 0.15419 nm.

In still another embodiment, the present invention relates to a crystalline form (Form HyB) of belumosudil mesylate characterized by having a PXRD comprising reflections at 2-Theta angles of (6.4±0.1)°, (6.6±0.1)°, (8.1±0.1)°, (12.9±0.1)°, (15.9±0.1)°, (16.3±0.1)°, (19.5±0.1)°, (19.8±0.1)°, (26.3±0.1)° and (26.4±0.1)°, when measured at a temperature in the range of from 20 to 30° C. with Cu-Kalpha_{1,2} radiation having a wavelength of 0.15419 nm.

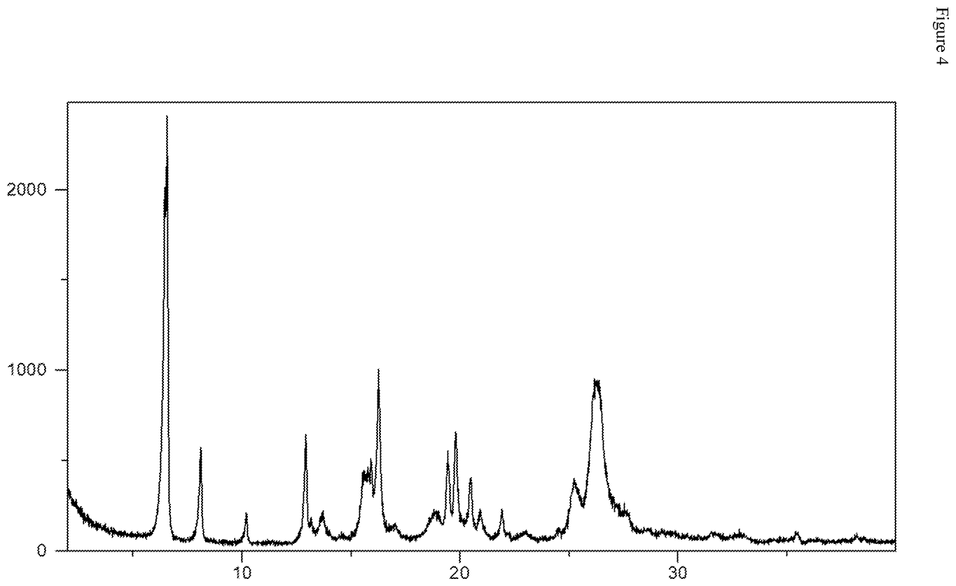

In yet another embodiment, the present invention relates to a crystalline form (Form HyB) of belumosudil mesylate characterized by having a PXRD essentially the same as shown in FIG. 4 of the present invention, when measured at a temperature in the range of from 20 to 30° C. with Cu-Kalpha_{1,2} radiation having a wavelength of 0.15419 nm.

In another embodiment, the present invention relates to a crystalline form (Form HyB) of belumosudil mesylate, characterized by having a DSC curve comprising an endothermic peak, having an onset at a temperature of about (75±5)° C., preferably of about (75±3)° C., more preferably of about (75±1)° C., such as about 74.7° C. when measured at a heating rate of 10 K/min.

In a further embodiment, the present invention relates to a crystalline form (Form HyB) of belumosudil mesylate as defined in any one of the above described embodiments characterized in being a monohydrate.

### Amorphous Belumosudil Mesylate

In another aspect the present invention relates to amorphous belumosudil mesylate according to the chemical structure as depicted in Formula (B)

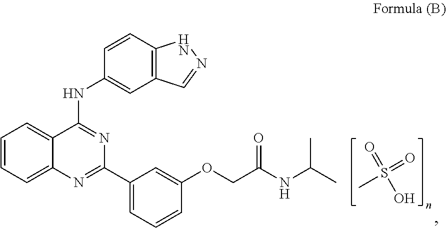

wherein n is in the range of from 0.8 to 1.2, preferably of from 0.9 to 1.1, even more preferably of from 0.95 to 1.05 and most preferably n is 1.0.

In one embodiment, the present invention relates to amorphous belumosudil mesylate characterized by having a PXRD comprising no reflections, when measured at a temperature in the range of from 20 to 30° C. with Cu-Kalpha_{1,2} radiation having a wavelength of 0.15419 nm.

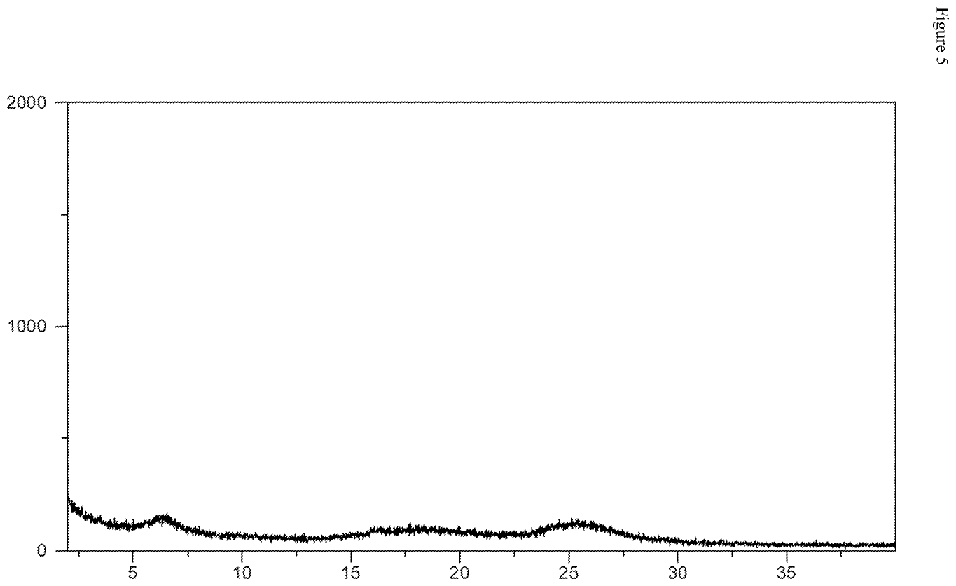

In yet another embodiment, the present invention relates to amorphous belumosudil mesylate characterized by having a PXRD essentially the same as shown in FIG. 5 of the present invention, when measured at a temperature in the range of from 20 to 30° C. with Cu-Kalpha_{1,2} radiation having a wavelength of 0.15419 nm.

### Pharmaceutical Compositions and Medical Use

In a further aspect, the present invention relates to the use of a crystalline belumosudil mesylate form (e.g. Form I, II, HyA or HyB) or amorphous belumosudil mesylate of the present invention as defined in any one of the above described aspects and their corresponding embodiments for the preparation of a pharmaceutical composition.

Furthermore, the present invention relates to a pharmaceutical composition comprising a crystalline belumosudil mesylate form (e.g. Form I, II, HyA or HyB) or amorphous belumosudil mesylate of the present invention as defined in any one of the above described aspects and their corresponding embodiments, preferably in an effective and/or predetermined amount and at least one pharmaceutically acceptable excipient.

Preferably, the effective and/or predetermined amount of the crystalline belumosudil mesylate form (e.g. Form I, II, HyA or HyB) or amorphous belumosudil mesylate of the present invention as defined in any one of the above described aspects and their corresponding embodiments is in the range of from about 10 to 200 mg, calculated as belumosudil. For example, the effective and/or predetermined amount is selected from the group consisting of 10 mg, 20 mg, 30 mg, 40 mg, 50 mg, 60 mg, 70 mg, 80 mg, 90 mg, 100 mg, 110 mg, 120 mg, 130 mg, 140 mg, 150 mg, 160 mg, 170 mg, 180 mg, 190 mg and 200 mg calculated as belumosudil. Preferably, the effective and/or predetermined amount is 200 mg calculated as belumosudil.

The at least one pharmaceutically acceptable excipient, which is comprised in the pharmaceutical composition of the present invention, is preferably selected from the group consisting of fillers, disintegrants, binders, lubricants, glidants and any combinations thereof. Preferably, the at least one pharmaceutically acceptable excipient, which is comprised in the pharmaceutical composition of the present invention is selected from the group consisting of microcrystalline cellulose, hypromellose, croscarmellose sodium, colloidal silicon dioxide, magnesium stearate and any combinations thereof.

Preferably, the pharmaceutical composition of the present invention is an oral solid dosage form, more preferably a tablet or a capsule. In a particular preferred embodiment, the pharmaceutical composition of the present invention as described above is a film-coated tablet, or a hard gelatin capsule, most preferably a film-coated tablet.

In another embodiment the present invention is directed at a film-coated tablet comprising a tablet core and a film coating, wherein the tablet core comprises 200 mg of a crystalline belumosudil mesylate form (e.g. Form I, II, HyA or HyB) or amorphous belumosudil mesylate of the present invention as defined in any one of the above described aspects and their corresponding embodiments calculated as belumosudil, microcrystalline cellulose, hypromellose, croscarmellose sodium, colloidal silicon dioxide and magnesium stearate. In a particular embodiment, the film-coating is a non-functional film coating. In another embodiment, the film-coating is a polyvinyl alcohol based film coating. For example, the film-coating comprises polyvinyl alcohol, polyethylene glycol, talc, titanium dioxide and iron oxide.

The pharmaceutical composition of the present invention as defined in any one of the above described embodiments may be produced by standard manufacturing processes, which are well-known to the skilled person e.g. selected from the group consisting of micronization, blending, milling, granulation (wet or dry granulation), capsule filling, tabletting, film-coating and any combinations thereof.

In a further aspect, the present invention relates to a crystalline belumosudil mesylate form (e.g. Form I, II, HyA or HyB) or amorphous belumosudil mesylate of the present invention or a pharmaceutical composition comprising a crystalline belumosudil mesylate form (e.g. Form I, II, HyA or HyB) or amorphous belumosudil mesylate of the present invention as defined in any one of the above described aspects and their corresponding embodiments for use as a medicament.

In still a further aspect, the present invention relates to a crystalline belumosudil mesylate form (e.g. Form I, II, HyA or HyB) or amorphous belumosudil mesylate of the present invention or a pharmaceutical composition comprising a crystalline belumosudil mesylate form (e.g. Form I, II, HyA or HyB) or amorphous belumosudil mesylate of the present invention as defined in any one of the above described aspects and their corresponding embodiments for use in the treatment and/or prophylaxis of an autoimmune disease.

In another aspect, the present invention relates to a method of treating an autoimmune disease said method comprising administering an effective amount of a crystalline belumosudil mesylate form (e.g. Form I, II, HyA or HyB) or amorphous belumosudil mesylate of the present invention or a pharmaceutical composition comprising a crystalline belumosudil mesylate form (e.g. Form I, II, HyA or HyB) or amorphous belumosudil mesylate of the present invention as defined in any one of the above described aspects and their corresponding embodiments to a patient in need of such a treatment.

In a preferred embodiment, the autoimmune disease is graft-versus-host disease (GvHD) or systemic sclerosis. In a particular preferred embodiment, the autoimmune disease is graft-versus-host disease.

### EXAMPLES

The following non-limiting examples are illustrative for the disclosure and are not to be construed as to be in any way limiting for the scope of the invention. Crude belumosudil can be obtained by following the procedure disclosed in example 82 of WO 2006/105081 A1.

### Example 1: Preparation of Belumosudil Mesylate Form I

To a suspension of belumosudil (202.2 mg) in methanol (4 mL) methanesulfonic acid (40 μl, 1.4 eq) was added. The obtained solution was left standing at room temperature for 4 hours before the obtained crystals were collected by filtration and dried, first under vacuum (about 5 mbar) at room temperature overnight (about 18 hours) and then under vacuum (about 5 mbar) at 65° C. over the weekend to yield belumosudil mesylate form I (223.7 mg).

### Example 2: Preparation of Belumosudil Mesylate Form II

Belumosudil mesylate form HyA (216 mg, e.g. prepared in analogy to the procedure disclosed in example 3 herein) was annealed at 230° C. on a heat plate for 15 minutes and thereafter cooled to room temperature to yield belumosudil mesylate form II (197 mg).

### Example 3: Preparation of Belumosudil Mesylate Form HyA

To a suspension of belumosudil (204.5 mg) in ethanol (4 mL; 75% v/v) methanesulfonic acid (40 μl, 1.4 eq) was added. The suspension was stirred at room temperature for 1 hour before the obtained crystals were collected by filtration and dried under vacuum (about 5 mbar) at room temperature for 4 hours to yield belumosudil mesylate form HyA (194.9 mg).

### Example 4: Preparation of Belumosudil Mesylate Form HyB

To a suspension of belumosudil (50.0 mg) in ethanol (1 mL; 75% v/v) methanesulfonic acid (10 μl, 1.4 eq) was added. The suspension was left standing at room temperature for 1 hour before the obtained crystals were collected by centrifugation and dried under vacuum (about 5 mbar) at room temperature for 2.5 hours to yield belumosudil mesylate form HyB.

### Example 5: Preparation of Amorphous Belumosudil Mesylate

To a suspension of belumosudil (50 mg) in water (10 mL) methanesulfonic acid (10 μl, 1.4 eq) and additional water (5 mL) were added and the obtained mixture was stirred at room temperature overnight (about 18 hours). Acetonitrile (8 mL) was added and the obtained solution was lyophilized to yield amorphous belumosudil mesylate quantitatively.

### Example 6: Powder X-Ray Diffraction

Powder X-ray diffraction was performed with a PANalytical X'Pert PRO diffractometer equipped with a theta/theta coupled goniometer in transmission geometry, Cu-Kalpha_{1,2} radiation (wavelength 0.15419 nm) with a focusing mirror and a solid state PIXcel detector. Diffractograms were recorded at a tube voltage of 45 kV and a tube current of 40 mA, applying a stepsize of 0.013° 2-theta with 40 s per step (255 channels) in the angular range of 2° to 40° 2-Theta at ambient conditions. A typical precision of the 2-Theta values is in the range of ±0.2° 2-Theta, preferably of ±0.1° 2-Theta.

A representative diffractogram of belumosudil mesylate Form I is displayed in FIG. 1 hereinafter. The corresponding reflection list in the range of from 2 to 30° 2-Theta is provided in Table 1 below.

TABLE 1
Reflection positions of crystalline Form I of belumosudil mesylate in
the range of from 2 to 30° 2-Theta; a typical precision of the 2-Theta
values is in the range of ±0.2° 2-Theta, preferably of ±0.1° 2-Theta.
Reflection position [°2-Theta]

7.1
8.4
12.5
13.0
13.4
13.8
14.1
14.5
15.3
15.7
16.1
16.5
16.8
17.3
18.4
19.5
20.3
21.0
21.6
22.2
22.9
23.6
24.6
25.1
25.6
26.1
26.6
27.3
27.6
28.3
29.1

A representative diffractogram of belumosudil mesylate Form II is displayed in FIG. 2 hereinafter. The corresponding reflection list in the range of from 2 to 30° 2-Theta is provided in Table 2 below.

TABLE 2
Reflection positions of crystalline Form II of belumosudil mesylate in
the range of from 2 to 30° 2-Theta; a typical precision of the 2-Theta
values is in the range of ±0.2° 2-Theta, preferably of ±0.1° 2-Theta.
Reflection position [°2-Theta]

6.9
7.3
9.4
12.2
12.8
13.3
13.7
13.9
14.6
15.3
15.6
16.6
17.4
18.0
18.6
19.0
19.6
20.5
20.9
21.0
21.7
21.9
22.4
23.0
24.1
24.7
25.3
25.9
26.8
27.3
27.6
28.9
29.4

A representative diffractogram of belumosudil mesylate Form HyA is displayed in FIG. 3 hereinafter. The corresponding reflection list in the range of from 2 to 30° 2-Theta is provided in Table 3 below.

TABLE 3
Reflection positions of crystalline Form HyA of belumosudil mesylate in
the range of from 2 to 30° 2-Theta; a typical precision of the 2-Theta
values is in the range of ±0.2° 2-Theta, preferably of ±0.1° 2-Theta.
Reflection position [°2-Theta]

6.3
7.9
10.0
12.7
13.6
13.9
14.5
15.5
15.8
16.0
16.9
19.0
20.0
20.4
21.5
23.8
25.2
25.4
26.5
27.4
29.3

A representative diffractogram of belumosudil mesylate Form HyB is displayed in FIG. 4 hereinafter. The corresponding reflection list in the range of from 2 to 30° 2-Theta is provided in Table 4 below.

TABLE 4
Reflection positions of crystalline Form HyB of belumosudil mesylaste in
the range of from 2 to 30° 2-Theta; a typical precision of the 2-Theta
values is in the range of ±0.2° 2-Theta, preferably of ±0.1° 2-Theta.
Reflection position [°2-Theta]

6.4
6.6
8.1
10.2
12.9
13.7
15.6
15.9
16.3
17.0
18.8
19.5
19.8
20.5
20.9
21.9
23.0
24.5
25.3
26.3
26.4
27.6

A representative diffractogram of amorphous belumosudil mesylate is displayed in FIG. 5 hereinafter. As can be seen from FIG. 5 amorphous belumosudil mesylate possesses no distinct peak in its powder X-ray diffractogram proofing the absence of long range order which is characteristic for an amorphous material.

### Example 7: Differential Scanning Calorimetry

DSC was performed on a Mettler Polymer DSC R instrument. The samples (2.89 mg Form I, 5.18 Form II, 5.33 mg Form HyA and 5.84 mg Form HyB) were each heated in a 40 microliter aluminium pan with a pierced aluminium lid from 25 to 300° C. at a rate of 10° K/min. Nitrogen (purge rate 50 mL/min) was used as purge gas.

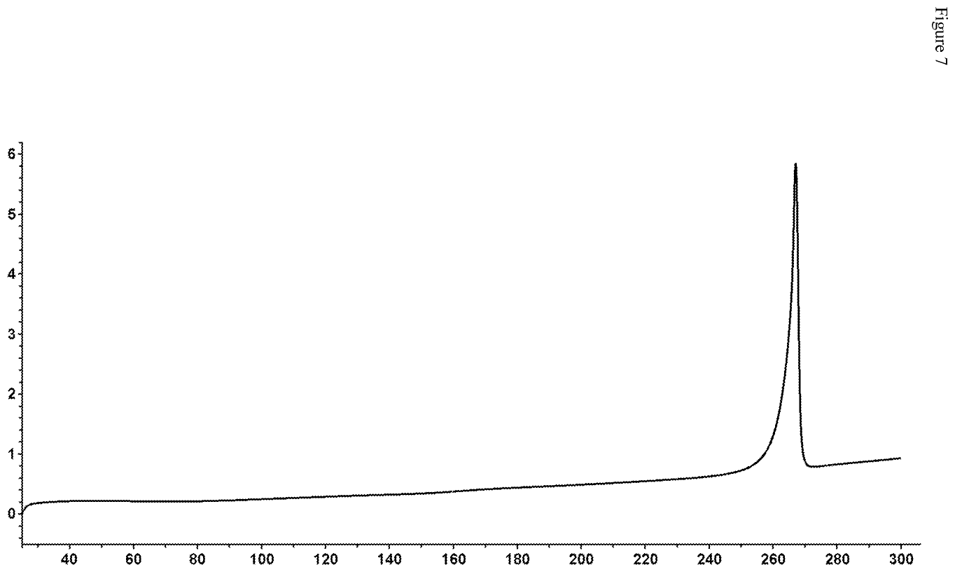

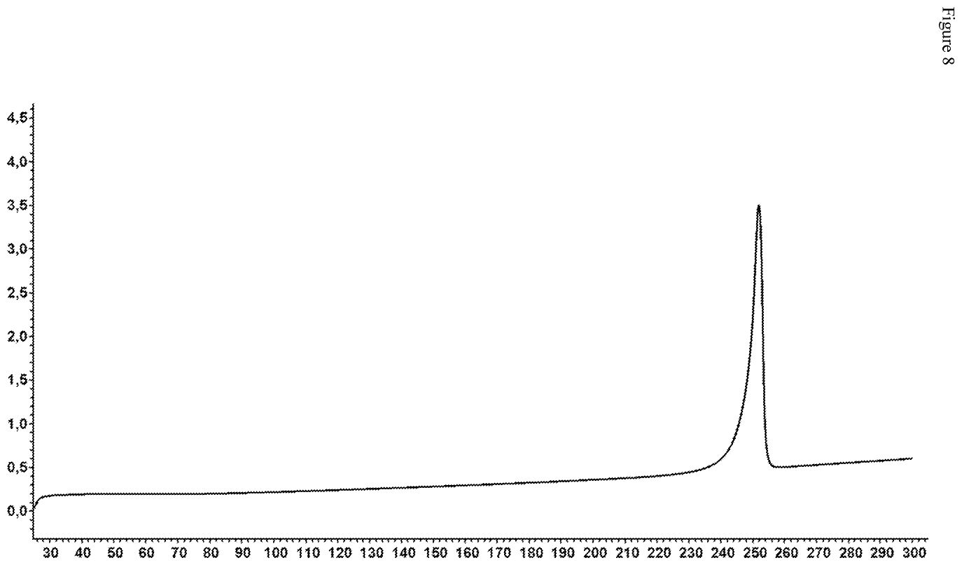

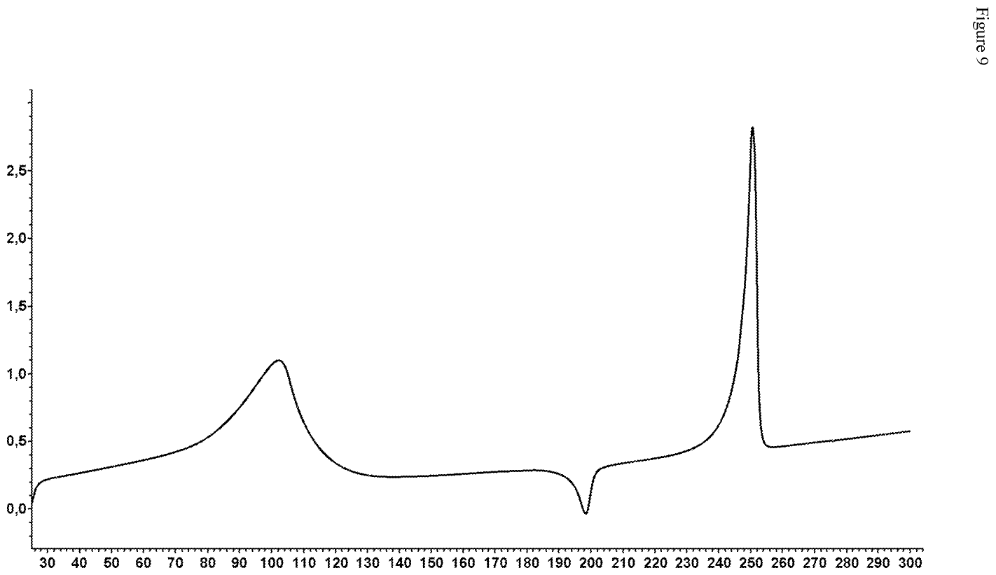

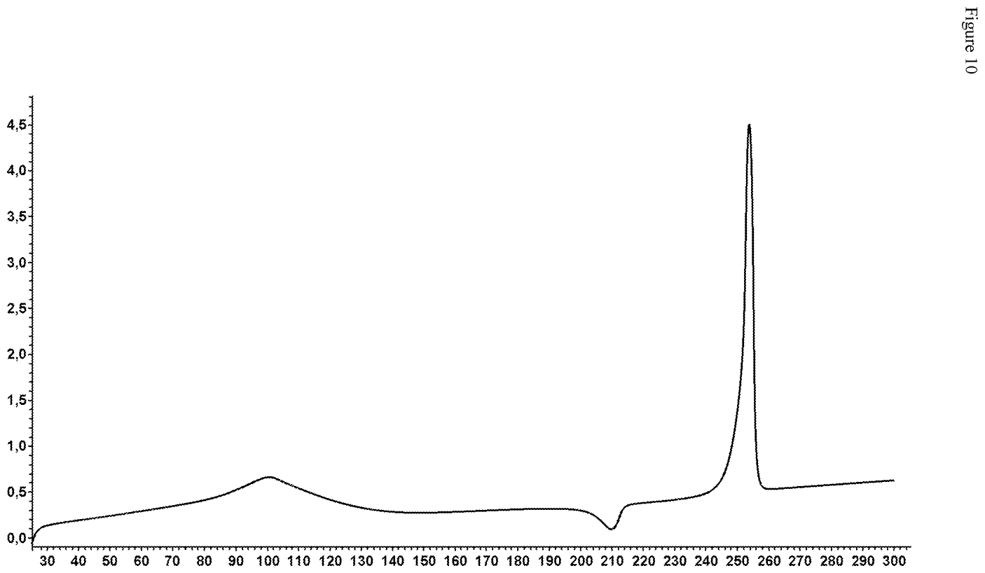

Representative DSC curves of the various belumosudil meslylate forms of the present invention are displayed in FIGS. 7 to 10 and the results are summarized in table 5 below.

TABLE 5
Thermal events during DSC experiments with
various forms of belumosudil mesylate

| Belumosudil | |
|---|---|
| mesylate | Thermal event |
| Form I | Endotherm: melting, Tₒₙₛₑₜ = 263.9° C., Tₚₑₐₖ = 266.0° C. |
| Form II | Endotherm: melting, Tₒₙₛₑₜ = 247.5° C., Tₚₑₐₖ = 250.8° C. |
| Form HyA | 1^{st} broad endotherm: dehydration, Tₒₙₛₑₜ = 76.3° C., |
| | Tₚₑₐₖ = 101.9° C. |
| | Exotherm: recrystallization of Form II, Tₒₙₛₑₜ = 193.6° C., |
| | Tₚₑₐₖ = 198.3° C., |
| | 2^{nd} endotherm: melting of Form II, Tₒₙₛₑₜ = 246.1° C., |
| | Tₚₑₐₖ = 249.7° C. |
| Form HyB | 1^{st} broad endotherm: dehydration, Tₒₙₛₑₜ = 74.7° C., |
| | Tpeak = 100.5° C. |
| | Exotherm: recrystallization of Form II, Tₒₙₛₑₜ = 202.4° C., |
| | Tₚₑₐₖ = 209.8° C., |
| | 2^{nd} endotherm: melting of Form II, Tₒₙₛₑₜ = 250.5° C., |
| | Tₚₑₐₖ = 252.2° C. |

### Example 8: Thermogravimetric Analysis

TGA was performed on a Mettler TGA/DSC 1 instrument. The samples (5.15 mg Form I, 5.69 Form II, 3.97 mg Form HyA, and 8.55 mg Form HyB) were each heated in a 100 microliter aluminium pan closed with an aluminium lid from 25 to 300° C. at a rate of 10 K/min. The lid was automatically pierced at the beginning of the measurement. Nitrogen (purge rate 50 mL/min) was used as purge gas.

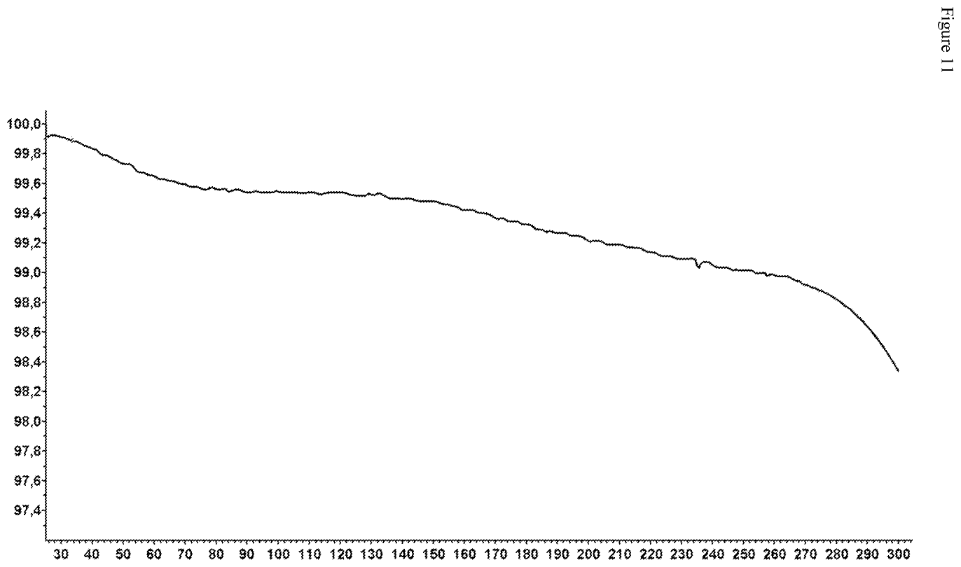

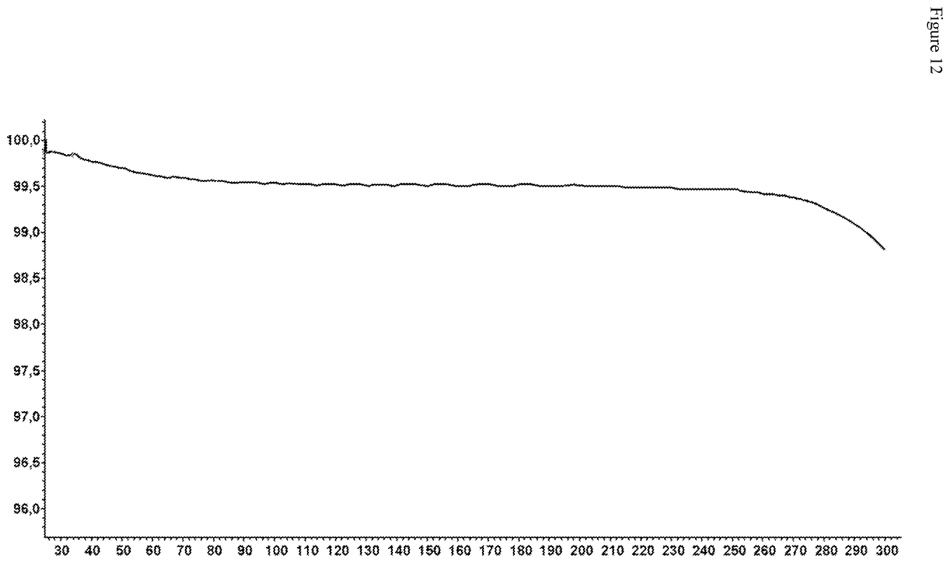

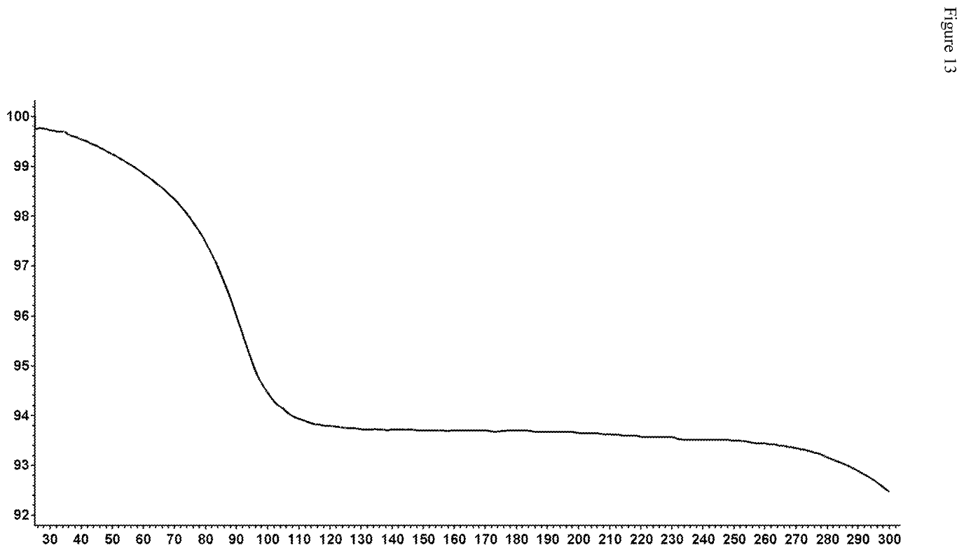

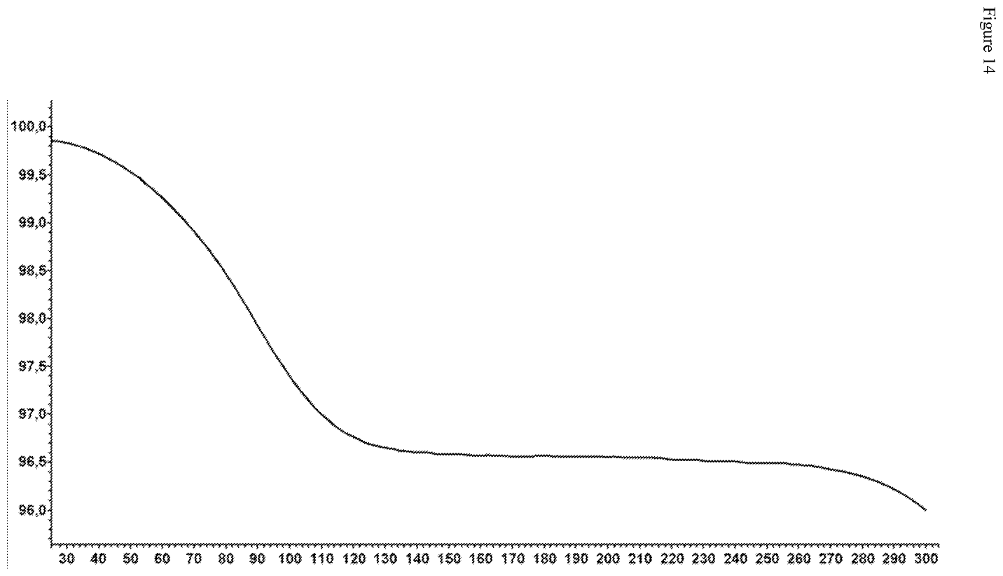

Representative TGA curves of the various belumosudil meslylate forms of the present invention are displayed in FIGS. 11 to 14 and the results are summarized in table 6 below.

TABLE 6
Mass losses during TGA experiments with
various forms of belumosudil mesylate

| Belumosudil | |
|---|---|
| mesylate | Thermal event |
| Form I | Δm (0-250° C.) = 0.87%, anhydrate |
| Form II | Δm (0-100° C.) = 0.32%, anhydrate |
| Form HyA | Δm (0-130° C.) = 5.97%, corresponds to a dihydrate |
| Form HyB | Δm (0-140° C.) = 3.19%, corresponds to a monohydrate |

### Reference Example 1: Preparation of Belumosudil HCl According to the Procedure Disclosed in Example 5 of WO 2014/055996 A1

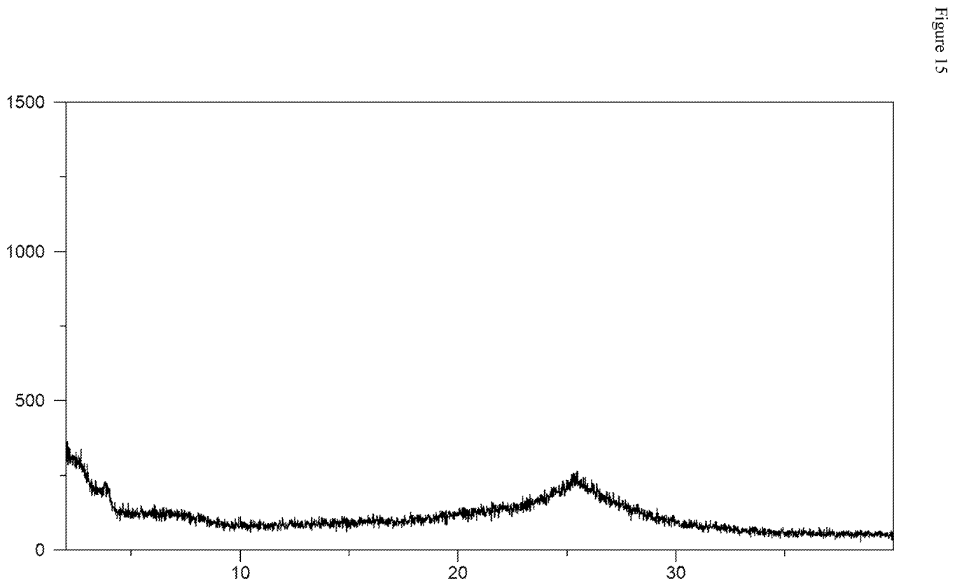

Belumosudil (215.4 mg) was taken up in 4 M HCl in dioxane (4 mL) and stirred at room temperature for 2 hours. Then the solvents were removed under vacuum to give belumosudil hydrochloride salt. The PXRD of the obtained sample is disclosed in FIG. 15 hereinafter.

### Reference Example 2: Preparation of Belumosudil Trifluoroacetate Salt According to the Procedure Disclosed in Example 92 of WO 2008/054599 A1

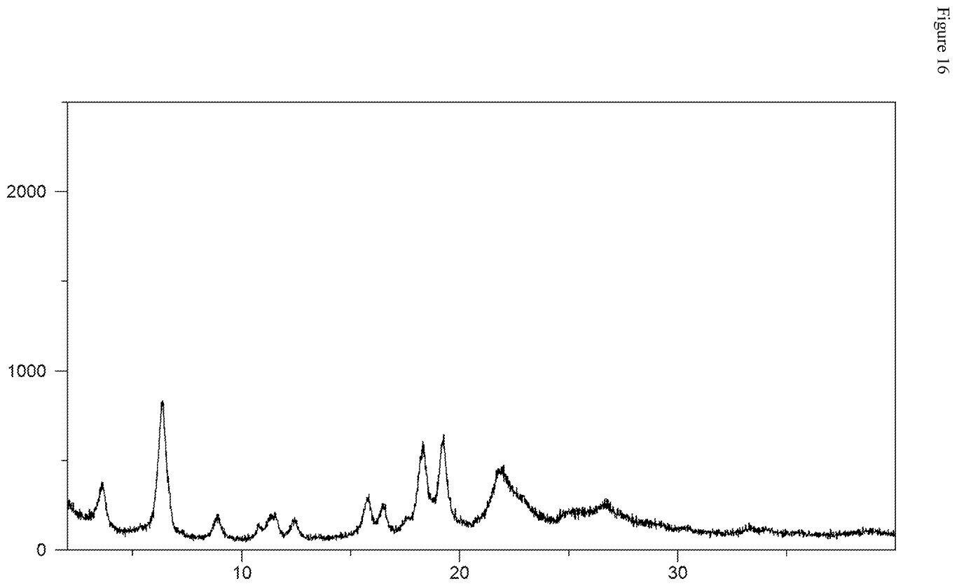

A solution of belumosudil (215.2 mg) in TFA (1 mL) and dichloromethane (1 mL) was stirred at RT for 1 hour. The solvents were removed under vacuum and diethyl ether (5 mL) was added to the residue. The mixture was stirred for about 20 minutes before the solid was collected by filtration and dried. The PXRD of the obtained sample is disclosed in FIG. 16 hereinafter.

### Comparative Example 1: Gravimetric Moisture Sorption

Moisture sorption isotherms were recorded with an SPSx-1μ moisture sorption analyzer (ProUmid, Ulm). The measurement cycle was started at ambient relative humidity (RH) of 40%. Relative humidity was then decreased to 5% RH in 5% steps, followed by a further decrease to 3% RH and to 0% RH. Afterwards RH was increased from 0% to 90% in a sorption cycle and decreased to 0% in a desorption cycle in 5% steps. Finally RH was increased from 0 to 40% in 5% steps.

The time per step was set to a minimum of 2 hours and a maximum of 6 hours. If an equilibrium condition with a constant mass of +0.01% within 1 hour was reached before the maximum time for all examined samples the sequential humidity step was applied before the maximum time of 6 hours. If no equilibrium was achieved the consecutive humidity step was applied after the maximum time of 6 hours. The temperature was 25±0.1° C.

The behaviour of the various sample in the range of 0 to 80% RH is summarized in Table 7 below:

TABLE 7
Behaviour of various belumosudil forms in the range
of 0-80% RH at (25 ± 0.1)° C.

| Belumosudil Form | Δm (0-80% RH) | Comment |
|---|---|---|
| Mesylate Form I | +1.9 w-% | Slightly hygroscopic |
| Mesylate Form II | +1.4 w-% | Slightly hygroscopic |
| Mesylate Form HyA | Not applicable | Dehydration below 3% RH |
| Mesylate Form HyB | Not applicable | Dehydration below 3% RH |
| Amorphous mesylate | +21.2 w % | Very hygroscopic |
| TFA salt | −12.7 w-% | Exchange of solvent vs. water |
| HCl salt | +14.3 w-% | Moderately hygroscopic |

As can be seen from Table 7 anhydrous belumosudil mesylate Form I and Form II can be assigned as being slightly hygroscopic, whereas amorphous belumosudil mesylate and belumosudil HCl prepared according to the procedure disclosed in Example 5 of WO 2014/055996 A1 take up significant amounts of water and therefore can be assigned as being moderately hygroscopic and very hygroscopic, respectively. In contrast, the TFA salt prepared according to the procedure disclosed in Example 92 of WO 2008/054599 A1 shows a significant weight loss of about 13% from 0 to 80% RH, indicating that organic solvent is present and exchanged against water at increasing relative humidity levels. Both hydrates of the belumosudil mesylate salt, Form HyA and Form HyB, release their crystal water only at very low relative humidities e.g. below 3% RH.

1. Crystalline belumosudil mesylate according to the chemical structure as depicted in Formula (B) 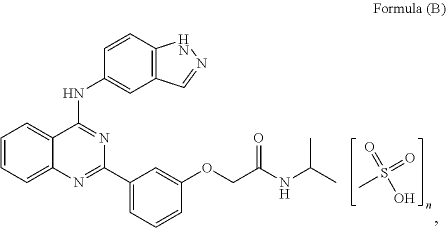
wherein n is in the range of from 0.8 to 1.2.

2. The crystalline belumosudil mesylate of claim 1 in crystalline Form I, characterized by having a powder X-ray diffractogram comprising reflections at 2-Theta angles of (7.1±0.2)°, (8.4±0.2)° and (25.6±0.2)°, when measured at a temperature in the range of from 20 to 30° C. with Cu-Kalpha_{1,2} radiation having a wavelength of 0.15419 nm.

3. The crystalline form of claim 2 characterized by having a differential scanning calorimetry curve comprising an endothermic peak with an onset at a temperature of (264±5)° C., when measured at a heating rate of 10K/min.

4. The crystalline belumosudil mesylate of claim 1 in crystalline Form II, characterized by having a powder X-ray diffractogram comprising reflections at 2-Theta angles of (7.3±0.2)°, (9.4±0.2)° and (19.6±0.2)°, when measured at a temperature in the range of from 20 to 30° C. with Cu-Kalpha_{1,2} radiation having a wavelength of 0.15419 nm.

5. The crystalline form of claim 4 characterized by having a differential scanning calorimetry curve comprising an endothermic peak with an onset at a temperature of (248±5)° C., when measured at a heating rate of 10K/min.

6. The crystalline belumosudil mesylate of claim 1 in crystalline Form HyA, characterized by having a powder X-ray diffractogram comprising reflections at 2-Theta angles of (6.3±0.2)°, (12.7±0.2)° and (14.5±0.2)°, when measured at a temperature in the range of from 20 to 30° C. with Cu-Kalpha_{1,2} radiation having a wavelength of 0.15419 nm.

7. The crystalline form of claim 6 characterized by having a differential scanning calorimetry curve comprising an endothermic peak with an onset at a temperature of (76±5)° C., when measured at a heating rate of 10K/min.

8. The crystalline belumosudil mesylate of claim 1 in crystalline Form HyB, characterized by having a powder X-ray diffractogram comprising reflections at 2-Theta angles of (6.6±0.2)°, (16.3±0.2)° and (19.5±0.2)°, when measured at a temperature in the range of from 20 to 30° C. with Cu-Kalpha_{1,2} radiation having a wavelength of 0.15419 nm.

9. The crystalline form of claim 8 characterized by having a differential scanning calorimetry curve comprising an endothermic peak with an onset at a temperature of (75±5)° C., when measured at a heating rate of 10K/min.

10. Process for preparing a pharmaceutical composition comprising the steps of providing the crystalline form as defined in claim 1; providing at least one pharmaceutically acceptable excipient; and obtaining said pharmaceutical composition.

11. A pharmaceutical composition comprising a crystalline form as defined in claim 1, and at least one pharmaceutically acceptable excipient.

12. The pharmaceutical composition according to claim 11, wherein the predetermined and/or effective amount is 200 mg calculated as belumosudil.

13. The pharmaceutical composition according to claim 12 which is an oral solid dosage form.

14. The pharmaceutical composition according to claim 12, wherein the oral solid dosage form is a tablet.

15. The pharmaceutical composition according to claim 14 wherein the tablet is a film-coated tablet.

16. A film-coated tablet comprising a tablet core and a film-coating, wherein the tablet core comprises 200 mg of a crystalline form of belumosudil mesylate as defined in claim 1 calculated as belumosudil, microcrystalline cellulose, hypromellose, croscarmellose sodium, colloidal silicon dioxide and magnesium stearate.

17. A method of treatment of a disease in a patient comprising administering to the patient in need of such a treatment a crystalline form as defined in claim 1.

18. The method according to claim 17, wherein the disease is an autoimmune disease.

19. The method according to claim 18, wherein the autoimmune disease is graft-versus-host disease (GvHD) or systemic sclerosis.

20. The pharmaceutical composition according to claim 11, wherein the crystalline belumosudil mesylate is characterized by having a powder X-ray diffractogram comprising reflections at 2-Theta angles of (7.1±0.2)°, (8.4±0.2)° and (25.6±0.2)°, when measured at a temperature in the range of from 20 to 30° C. with Cu-Kalpha_{1,2} radiation having a wavelength of 0.15419 nm.

21. The pharmaceutical composition according to claim 11, wherein the crystalline belumosudil mesylate is characterized by having a powder X-ray diffractogram comprising reflections at 2-Theta angles of (7.3±0.2)°, (9.4±0.2)° and (19.6±0.2)°, when measured at a temperature in the range of from 20 to 30° C. with Cu-Kalpha_{1,2} radiation having a wavelength of 0.15419 nm.

22. The pharmaceutical composition according to claim 11, wherein the crystalline belumosudil mesylate is characterized by having a powder X-ray diffractogram comprising reflections at 2-Theta angles of (6.3±0.2)°, (12.7±0.2)° and (14.5±0.2)°, when measured at a temperature in the range of from 20 to 30° C. with Cu-Kalpha_{1,2} radiation having a wavelength of 0.15419 nm.

23. The pharmaceutical composition according to claim 11, wherein the crystalline belumosudil mesylate is characterized by having a powder X-ray diffractogram comprising reflections at 2-Theta angles of (6.6±0.2)°, (16.3±0.2)° and (19.5±0.2)°, when measured at a temperature in the range of from 20 to 30° C. with Cu-Kalpha_{1,2} radiation having a wavelength of 0.15419 nm.

24. The film-coated tablet according to claim 16, wherein the tablet core comprises 200 mg of a crystalline form of belumosudil mesylate, characterized by having a powder X-ray diffractogram comprising reflections at 2-Theta angles of (7.1±0.2)°, (8.4±0.2)° and (25.6±0.2)°, when measured at a temperature in the range of from 20 to 30° C. with Cu-Kalpha_{1,2} radiation having a wavelength of 0.15419 nm, calculated as belumosudil.

25. The film-coated tablet according to claim 16, wherein the tablet core comprises 200 mg of a crystalline form of belumosudil mesylate, characterized by having a powder X-ray diffractogram comprising reflections at 2-Theta angles of (7.3±0.2)°, (9.4±0.2)° and (19.6±0.2)°, when measured at a temperature in the range of from 20 to 30° C. with Cu-Kalpha_{1,2} radiation having a wavelength of 0.15419 nm, calculated as belumosudil.

26. The film-coated tablet according to claim 16, wherein the tablet core comprises 200 mg of a crystalline form of belumosudil mesylate, characterized by having a powder X-ray diffractogram comprising reflections at 2-Theta angles of (6.3±0.2)°, (12.7±0.2)° and (14.5±0.2)°, when measured at a temperature in the range of from 20 to 30° C. with Cu-Kalpha_{1,2} radiation having a wavelength of 0.15419 nm, calculated as belumosudil.

27. The film-coated tablet according to claim 16, wherein the tablet core comprises 200 mg of a crystalline form of belumosudil mesylate, characterized by having a powder X-ray diffractogram comprising reflections at 2-Theta angles of (6.6±0.2)°, (16.3±0.2)° and (19.5±0.2)°, when measured at a temperature in the range of from 20 to 30° C. with Cu-Kalpha_{1,2} radiation having a wavelength of 0.15419 nm, calculated as belumosudil.
